(19) United States
(12) Patent Application Publication
(10) Pub. No.: US 2016/0303068 A1
(43) Pub. Date: Oct. 20, 2016

(54) Compositions and Methods For Treating Brain Injury and Brain Diseases

(71) Applicant: University of Washington through its Center for Commercialization, Seattle, WA (US)

(72) Inventors: Donna J. Cross, Seattle, WA (US); Satoshi Minoshima, Seattle, WA (US)

(73) Assignee: University of Washington through its Center for Co mmercialization, Seattle, WA (US)

(21) Appl. No.: 15/034,295

(22) PCT Filed: Nov. 7, 2014
(86) PCT No.: PCT/US14/64571

§ 371 Date: May 4, 2016

Related U.S. Application Data
(60) Provisional application No. 61/902,059, filed on Nov. 8, 2013.

(51) Int. Cl. A61K 31/337; A61K 9/00

(52) U.S. Cl. CPC A61K 31/337; A61K 9/0043

(57) ABSTRACT

The present disclosure provides methods of treating brain injuries or conditions, or symptoms of brain injuries or conditions, including intranasal administration of a therapeutically effective amount of a taxane. The intranasally administered taxane can bypass the blood-brain barrier (BBB) via the olfactory epithelium and thereby provide therapeutic effect to a subject.

### CROSS-REFERENCE TO RELATED APPLICATION

This application claims the benefit of U.S. Patent Application No. 61/902,059, filed Nov. 8, 2013, the disclosure of which is hereby incorporated by reference in its entirety.

### BACKGROUND

There are presently 1.7 million traumatic brain injuries (TBIs) that occur per year in the United States, with TBIs being the leading cause of death of people between 1 and 45 years of age. Widespread use of improvised explosive devices (IEDs) against the United States military has also resulted in approximately 17 percent of veterans reporting persistent cognitive deficits and post-concussive symptoms years after blast-TBI.

TBI can lead to neurodegenerative diseases. For example, repetitive mild TBI (mTBI) common in the sport of boxing can lead to a dementia syndrome that includes

Parkinson's disease-like motor signs and cognitive symptoms that include bradyphrenia (slowed thinking), confusion, and memory impairment. Chronic mTBI experienced by football players is associated with chronic traumatic encephalopathy (CTE) in mid-life that is evidenced by diffuse neurofibrillary tangles—hallmark pathologic brain injuries observed in several other neurodegenerative diseases. In addition, both moderate and severe head injuries significantly increase the risk of developing Alzheimer's disease (AD), and head trauma poses the greatest known environmental risk factor for development of Alzheimer's disease. In mice, mild repetitive, but not single mild TBI episodes, increased Aβ (a molecule that plays a critical role in AD pathogenesis) and disturbed memory. Such data suggest a causal link between TBI and AD. To date, no effective pharmacological interventions exist to improve patient outcome following TBI.

Cytoskeletal disruption and axonal transport dysfunction are the most immediate consequences in mild to moderate concussive brain injury. Specifically, immediately following trauma, brain axonal fibers become misaligned and transport processes are disrupted, which can occur in a cascade of metabolic and neuroinflammatory events. If the damage is relatively mild, the brain's repair mechanisms can correct the injury with time. However, if the injury is more severe, or there are repeat injuries that occur prior to full recovery, then the cytoskeleton and transport mechanisms may become permanently impaired. This damage can lead to neuronal degeneration and loss, and long term deficits in cognitive functions. Indeed, the post mortem studies for pathological evidence of CTE of brains from athletes and military veterans who had suffered multiple mild TBIs found tau protein to accumulate in areas that are most vulnerable to impact-related shearing, such as near vessels and at the depths of the sulci even in young brains (age of 20 years).

The link between traumatic brain injury and CTE is illustrated in FIG. 1. FIG. 2 shows the fractional anisotropy maps from veterans (n=15) with multiple blast TBIs, compared statistically to non-blast veteran controls (n=12) on a voxelwise basis (NEUROSTAT). Referring to FIG. 2, diffusion tensor imaging (DTI) evidence indicated persistent damage to white matter tracts in the brains of blast-exposed veterans 3-5 years from last mild TBI, specifically to both anterior and posterior corpus callosum 3-5 years after mTBI (Z≧4.0). This loss of white matter structural integrity (assessed by DTI) was strongly associated with hypometabolic regions in normal aging and patients with mild cognitive impairment (MCI).

In addition to these findings of structural impairments in both TBI and neurodegeneration and the impact of such impairment on brain metabolic function, there have been studies indicating that disruption of axonal transport is both a consequence of TBI and an early feature in the pathogenesis of AD and other dementias. These data suggest a causal link between initial injury and cytoskeletal disruption leading to chronic loss of white matter integrity and a possible acceleration of the neurodegenerative cascade.

Microtubules are the main fibers that make up the axon cytoskeleton. Microtubule-stabilizing drugs have been proposed to be useful for treatment of Alzheimer's disease (AD) and other tauopathies, because it is believed that these drugs can ameliorate a loss of normal microtubule stabilization (due to disengagement of hyperphosphorylated tau protein in AD from microtubules), which can lead to a perturbation of neuronal functions including decreased axonal transport and overall loss of cytoskeletal integrity. The link between tau-protein hyperphosphorylation and bundling and AD and Alzheimer-related tauopathies is illustrated in FIG. 3. One such drug, epothilone D, which has blood-brain barrier penetration, was shown to improve cognitive performance and reduce associated tau pathology in AD transgenic mouse models. However, human applications of epothilone D for AD were limited by concerns of intravenous administration and side effects resulting from P-glycoprotein (Pgp) transporter inhibition over prolonged (e.g., decades) of dosing.

A class of microtubule-stabilizing drugs, known as taxanes, is already FDA approved for chemotherapy of some cancers (e.g., breast and lung) and inhibits mitosis by stabilizing microtubules. Recently, paclitaxel (i.e., taxol) was shown to facilitate axon regeneration after spinal cord injury by promoting axonal stabilization and decreasing Wallerian degeneration. As a class of drugs that has been well characterized as an anti-cancer therapeutic, taxanes (including paclitaxel) have the advantage in that their pharmacokinetics, pharmacodynamics, effective therapeutic window and side effects are well understood. “Druggability” in terms of target characterization and the availability of biological assays is also well established. Taxanes (such as paclitaxel) bind to β tubulin on the inner surface of the microtubule and counteracts the effects of guanosine-5′-triphosphate (GTP) hydrolysis, thereby preventing depolymerization. Biological assay methods for paclitaxel activity in tissues include [³H]-paclitaxel, [¹⁸F]-fluoropaclitaxel and LC-MS/MS quantitative analyses methods. Neurotherapeutic effects from paclitaxel administration have also been investigated in Adlard, P. A. et al., Acta Neuropathol., 2000. 100(2): p. 183-8; Hellal, F. et al., Science, 2011. 331(6019): p. 928-31; and Michaelis, M. L. et al., J. Mol. Neurosci., 2002. 19(3): p. 289-93. Despite these advantages, taxanes do not easily cross the blood-brain barrier and for this reason, they are considered unsuitable for treatment of brain tissue. Furthermore, there may be undesirable side effects to systemic administration of taxanes.

Thus, there is a need for methods of administering taxanes to the brain to relieve neuroinflammation, for example, following brain injury, or for the treatment of Alzheimer's disease and Alzheimer's related tauopathies. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a method of increasing microtubule stabilization in a brain tissue of a subject, including intranasally administering to the subject a therapeutically effective amount of a taxane.

In yet another aspect, the present disclosure features a method of decreasing tau protein oligomerization in a brain tissue of a subject, including intranasally administering to the subject a therapeutically effective amount of a taxane.

In yet another aspect, the present disclosure features a method of decreasing aggregation of a hyperphosphorylated tau protein in a brain tissue of a subject, including intranasally administering to a subject a therapeutically effective amount of a taxane.

In yet another aspect, the present disclosure features a method of ameliorating a condition having decreased microtubule stabilization in a brain tissue of a subject, including intranasally administering to the subject a therapeutically effective amount of a taxane.

In yet another aspect, the present disclosure features a method of ameliorating a condition having tau protein oligomerization in a brain tissue of a subject, including intranasally administering to the subject a therapeutically effective amount of a taxane.

In yet another aspect, the present disclosure features a method of ameliorating a condition having a hyperphosphorylated tau protein in a brain tissue of a subject, including intranasally administering to a subject a therapeutically effective amount of a taxane.

In yet another aspect, the present disclosure features a method of ameliorating a condition having neuroinflammation in a brain tissue of a subject, including intranasally administering to a subject a therapeutically effective amount of a taxane.

In yet another aspect, the present disclosure features a method of treating Alzheimer's disease in a subject, including intranasally administering to the subject a therapeutically effective amount of a taxane.

In yet another aspect, the present disclosure features a method of treating an Alzheimer-related tauopathy in a subject, including intranasally administering to the subject a therapeutically effective amount of a taxane.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a diagram showing the relationship between traumatic brain injury and chronic traumatic encephalopathy;

FIG. 2 is a diffusion tensor image of brains of blast-exposed veterans 3-5 years from last mild TBI compared to non-blast veteran controls;

FIG. 3 is a diagram showing the relationship between tau-protein hyperphosphorylation and bundling to Alzheimer's disease and Alzheimer-related tauopathies;

FIGS. 4 A and 4B are graphs showing [³H] paclitaxel distribution via intranasal administration in male CD-1 mice, 8 weeks, after administration of 1 μCi of [³H] paclitaxel in 1 μl PBS to the right nares;

FIG. 5 is a flow chart of a general protocol for evaluating intranasal treatment Alzheimer's disease, or Alzheimer-related tauopathies, with taxanes;

FIG. 6 is a graph showing brain injury volume for a rodent model of traumatic brain injury that has been treated with saline or paclitaxel;

FIG. 7A is an image of thresholded T2-maps for quantification of edema in a rodent model of traumatic brain injury, where the arrows point to regions of injury in saline and paclitaxel-treated subjects;

FIG. 7B is a graph showing injury-associated edema size for a rodent model of traumatic brain injury that has been treated with saline or paclitaxel;

FIG. 8A is a macromolecular proton fraction image of a rodent model of traumatic brain injury;

FIG. 8B is a graph showing bound pool fractions for a rodent model of traumatic brain injury that has been treated with saline or paclitaxel;

FIG. 9A is a graph showing maximum print area for a rodent model of traumatic brain injury that has been treated with saline or paclitaxel, when subjected to gait analysis;

FIG. 9B is a graph showing a mean intensity for a rodent model of traumatic brain injury that has been treated with saline or paclitaxel, when subjected to gait analysis;

FIG. 10 is a graph showing transport rates in transgenic mice treated with paclitaxel compared to saline controls;

FIG. 11 is a confocal microscopy image of sections from CA1 hippocampus of a rodent model of Alzheimer's disease that has been stained for astrocytes;

FIG. 12A is a graph showing normalized MRI intensity the T1-weighted images (normalized to global values to account for scanner drift between scans) vs. time in transgenic mice, after treatment with paclitaxel; and

FIG. 12B is a graph showing relative axonal transport rates in transgenic and wild-type mice treated with paclitaxel.

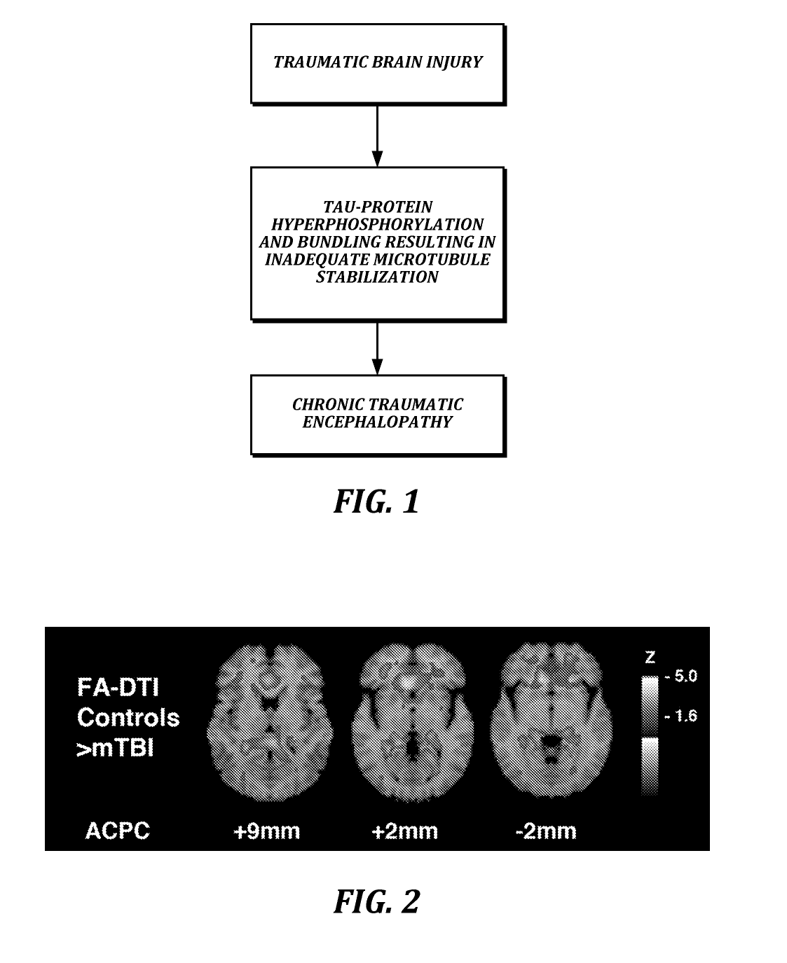

### DETAILED DESCRIPTION

The present disclosure provides methods of treating brain injuries or conditions, or symptoms of brain injuries or conditions, including intranasal administration of a therapeutically effective amount of a taxane. Provided herein are methods of increasing microtubule stabilization in a brain tissue, methods of decreasing tau protein oligomerization in a brain tissue, and methods of decreasing aggregation of a hyperphosphorylated tau protein in a brain tissue, including intranasally administering to a subject a therapeutically effective amount of a taxane.

Also provided herein are methods of ameliorating a condition having decreased microtubule stabilization in a brain tissue, methods of ameliorating a condition having tau protein oligomerization in a brain tissue, methods of ameliorating a condition having a hyperphosphorylated tau protein in a brain tissue, and methods of ameliorating a condition having neuroinflammation in a brain tissue, including intranasally administering to a subject a therapeutically effective amount of a taxane.

Also provided herein are methods of treating Alzheimer's disease, an Alzheimer-related tauopathy, or a traumatic brain injury in a subject, including intranasally administering to the subject a therapeutically effective amount of a taxane.

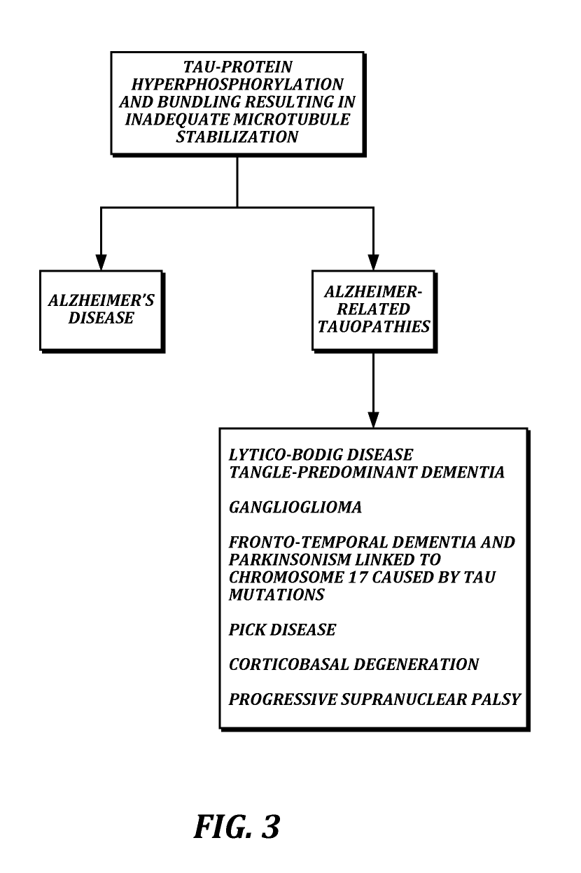

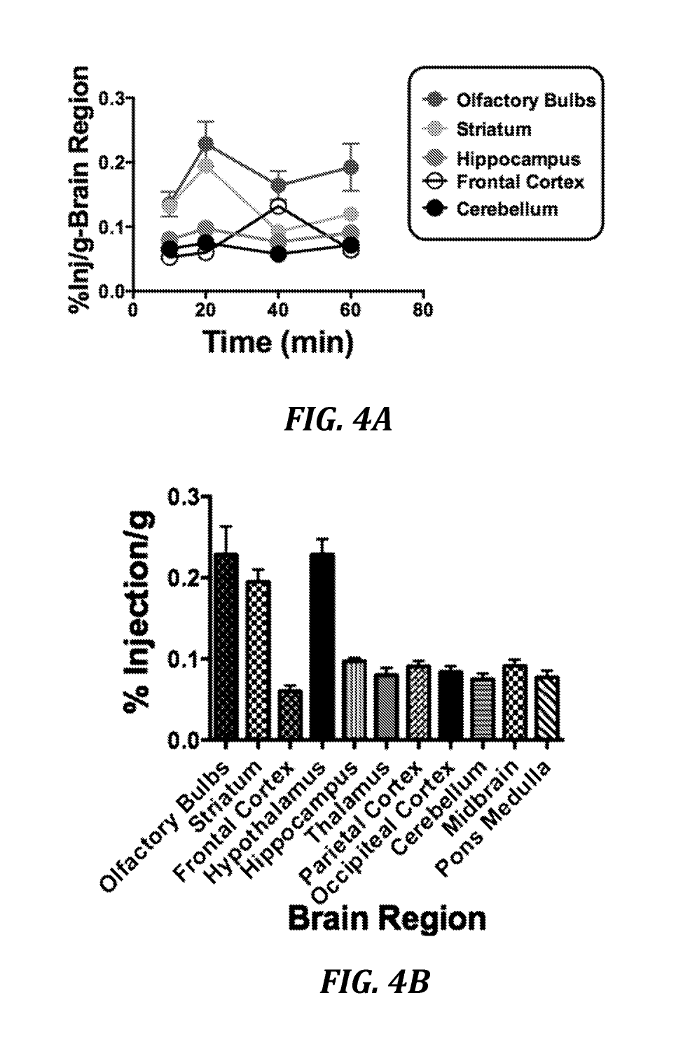

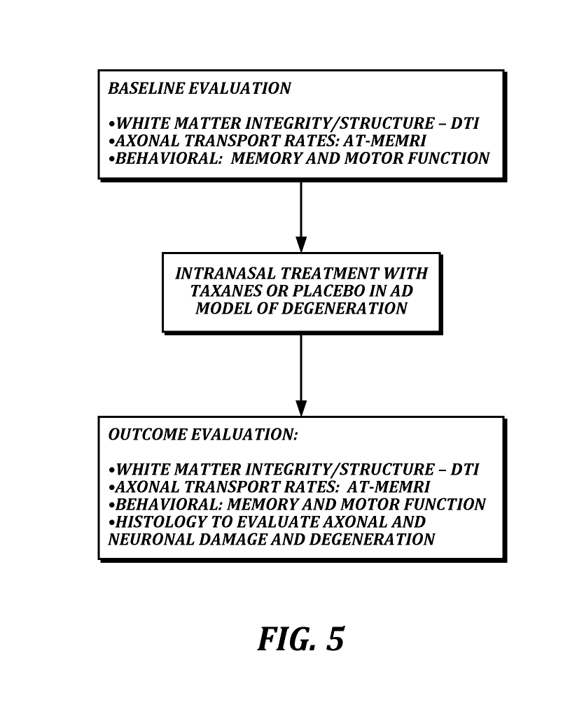

FIGS. 4A and 4B show distribution of a taxane (paclitaxel) in the brain, when the taxane is administered intranasally. Referring to FIG. 4A, taxane is taken up by the olfactory bulbs, the striatum, the hippocampus, the frontal cortex, the hypothalamus, and the cerebellum upon intranasal administration. Referring to FIG. 2, high striatum and hypothalamus uptake is observed after intranasal administration of the taxane. Thus, the intranasally administered taxane can bypass the blood-brain barrier (BBB) via the olfactory epithelium, and thereby provide therapeutic effect to a subject. The treatment methods can advantageously administer drug to the brain (e.g., an injured area of the brain) while minimizing side effects in non-target organs. The methods can have widespread applications. For example, intranasal administration of taxanes can result in decreased neuroinflammation, and can therefore be used in treatment of neuroinflammatory conditions, such as neurodegenerative diseases including Alzheimer's disease and related tauopathies, multiple sclerosis, viral infections, Parkinson's disease, as well as head trauma and mild concussions.

In some embodiments, the Alzheimer's disease and Alzheimer-related tauopathy treatable by the methods of the disclosure is each characterized by aggregation of a hyperphosphorylated tau protein in brain tissue into bundles of filaments. The Alzheimer-related tauopathy can include Lytico-Bodig disease, tangle-predominant dementia, ganglioglioma, fronto-temporal dementia and Parkinsonism linked to chromosome 17 (FTDP-17) caused by tau mutations, Pick disease, corticobasal degeneration, and/or progressive supranuclear palsy.

In some embodiments, the traumatic brain injury treatable by the methods of the disclosure includes skull fracture, brain swelling, penetrating skull injury, concussion, post-concussive symptoms, or any combination thereof. The concussion can result in loss of consciousness (e.g., for seconds, minutes, or for over 30 minutes). The post concussive symptoms can include headache, mental fog, decreased attention, decreased reaction time, concentration, sleep disturbance, mild motor disturbance, or any combination thereof. In some embodiments, intranasally administering a taxane is used to treat an acute traumatic brain injury. In some embodiments, intranasally administering a taxane is used to treat a chronic traumatic encephalopathy in a subject, which can result from repeated mild traumatic brain injuries, such as mild concussions.

In some embodiments, intranasally administering a therapeutically effective amount of a taxane can decrease the risk for an onset of Alzheimer's disease or chronic traumatic encephalopathy in a subject. For example, the subject may have been previously exposed to factors that may increase the risk of Alzheimer's disease or chronic traumatic encephalopathy, such as repeated mild concussions. Intranasal administration of a therapeutically effective amount of a taxane for a period following each mild concussion can decrease the likelihood of development of Alzheimer's disease or chronic traumatic encephalopathy.

### Definitions

It is appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, “traumatic brain injury” (TBI) refers to a form of acquired brain injury that occurs when a sudden trauma causes brain damage. TBI can occur when the head suddenly and violently hits an object, or when an object pierces the skull and enters brain tissue. TBI symptoms can be mild, moderate, or severe, depending on the extent of the damage to the brain.

Although the terms “mild,” “moderate,” or “severe” can be applied arbitrarily, generally, “mild” traumatic brain injury refers to a traumatic brain injury that results in loss of consciousness for a few seconds to a few minutes; no loss of consciousness, but a dazed, confused or disoriented state; headache; nausea or vomiting; fatigue or drowsiness; difficulty sleeping; sleeping more than usual; and/or dizziness or loss of balance. The mild traumatic brain injury can also create blurred vision; ringing in the ears; a bad taste in the mouth or changes in the ability to smell; and/or sensitivity to light or sound. Cognitive or mental symptoms of mild traumatic brain injury include memory or concentration problems; mood changes or mood swings; and/or feeling depressed or anxious. “Moderate” or “severe” traumatic brain injury refers to a traumatic brain injury that results in loss of consciousness from several minutes to hours; persistent headache or headache that worsens; repeated vomiting or nausea; convulsions or seizures; dilation of one or both pupils of the eyes; clear fluids draining from the nose or ears; inability to awaken from sleep; weakness or numbness in fingers and toes; and/or loss of coordination. Cognitive and mental symptoms include profound confusion; agitation; combativeness or other unusual behavior; slurred speech; coma and/or other disorders of consciousness.

As used herein, “Alzheimer-related tauopathy” refers to a class of neurodegenerative diseases associated with the pathological aggregation of tau protein in the brain. In Alzheimer-related tauopathies, tangles are formed by hyperphosphorylation of tau protein (a microtubule-associated protein), causing it to aggregate in an insoluble form. The aggregations of hyperphosphorylated tau protein are also referred to as paired helical filaments (PHF). Examples of Alzheimer-related tauopathy include Lytico-Bodig disease, tangle-predominant dementia, ganglioglioma, fronto-temporal dementia and Parkinsonism linked to chromosome 17 (FTDP-17) caused by tau mutations, Pick disease, corticobasal degeneration, and progressive supranuclear palsy.

As used herein, the term “modulate” is meant to refer to an ability to increase or decrease activity of an enzyme, a receptor, or a process. Modulation can occur in vitro or in vivo. Modulation can further occur in a cell.

As used herein, the term “cell” is meant to refer to a cell that is in vitro, ex vivo, or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” a taxane with a brain tissue includes the administration of a taxane to a brain of an individual, a subject or patient, such as a human, as well as, for example, introducing a taxane into a brain tissue sample.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of a taxane that elicits the biological or medicinal response that is being sought in a tissue, system, animal, individual or human by a researcher, veterinarian, medical doctor or other clinician, which includes one or more of the following:

(1) preventing the disease; for example, preventing a disease, condition or disorder in an individual who may be predisposed to the disease, condition or disorder but does not yet experience or display the pathology or symptomatology of the disease;

(2) inhibiting the disease; for example, inhibiting a disease, condition or disorder in an individual who is experiencing or displaying the pathology or symptomatology of the disease, condition or disorder; and

(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

### Taxane Mechanism of Action

Without wishing to be bound by theory, it is believed that taxanes can ameliorate a loss of normal microtubule stabilization, and thereby ameliorate neuroral functions due to decreased axonal transport and loss of cytoskeletal integrity. It is believed that that taxanes, such as paclitaxel (e.g., crystalline paclitaxel) and/or docetaxel, may also have modulatory effects on neuroinflammatory processes that could add to the overall therapeutic benefit in TBI. These effects may be attributed to microglial and astrocytic responses that are affected by MT-stabilization (proliferation and motility). Paclitaxel is also known to modulate estrogen receptor expression, which is present in microglia and astrocytes in the central nervous system. As microglia and astrocytes respond acutely to brain injury and these processes can be visualized in vivo with real time two-photon microscopy, the effect of paclitaxel administration on TBI-evoked neuroinflammation could be monitored using both histological and western blot analysis with in vivo response characterized by real time two-photon microscopy.

### Dosages

In some embodiments, intranasally administering a taxane includes administering taxane to a nasal passage (e.g., the epithelium of the nasal cavity, the epithelium of the upper nasal cavity, the superior nasal concha). In some embodiments, the taxane can be intranasally administered in the form of an aerosol, or an intranasal lavage. The taxane can include paclitaxel (e.g., crystalline paclitaxel) and/or docetaxel. The taxane can be in a formulation, which can include a pharmaceutically acceptable carrier.

The taxane can be administered in an amount of 0.1 mg/kg or more (e.g., 0.3 mg/kg or more, 0.5 mg/kg or more, 0.7 mg/kg or more, 1 mg/kg or more, 1.5 mg/kg or more) and/or about 2 mg/kg or less (e.g., 1.5 mg/kg or less, 1 mg/kg or less, 0.7 mg/kg or less, 0/5 mg/kg or less, or 0.3 mg/kg or less) per dose. In one embodiment, the taxane is administered in an amount of about 0.6 mg/kg per dose. The dose can be repeated at regular intervals, for example, every two weeks, every three weeks, every month, every two months, etc. In some embodiments, a total treatment period can last two weeks, a month, six months, a year, two years, or more. In some embodiments, in between periods of treatment, a subject can have a period during which no taxane is administered. In some embodiments, the amount of taxane that is administered can vary between doses.

The dosage can depend on variables such as the type and extent of progression of the disease or disorder, the overall health status of the particular patient, the relative biological efficacy of the taxane selected, and formulation of the excipient. Effective doses can be extrapolated from dose-response curves derived from in vitro or animal model test systems.

### Assays

The effectiveness of intranasal administration of taxanes in the methods of the present disclosure can be evaluated in a number of ways. Referring to Table 1, taxanes can improve a number of processes in the brain. These processes can each be evaluated using their corresponding parameters. The diseases that involve the improved processes are also listed. For example, improvement in neuroinflammation can be evaluated by immunostaining the CA1 region of the hippocampus of 3×Tg-AD mice, where reduced GFAP expression is present in paclitaxel treated mice compared to saline-treated mice. The improvement in neuroinflammation is important in the treatment of Alzheimer's disease and other conditions that evoke neuroinflammatory responses, such as Parkinson's disease, multiple sclerosis, certain viral infections (e.g., West Nile, herpes, HIV, and influenza), traumatic brain injury, and chronic traumatic encephalopathy.

TABLE 1
Processes that are improved by intranasal taxane administration, parameters for
their evaluation, and diseases involving the processes.

| Process improved by | | |
|---|---|---|
| taxanes | Evaluated parameters | Relevant diseases |
| Neuroinflammation | Immunostain in the CA1 | AD, other conditions that |
| (chronic) | region of the hippocampus | evoke neuroinflammatory |
| | of 3xTg-AD mice (7.5 mos.) | response such as |
| | showing reduced | Parkinson's disease, |
| | GFAP expression in | multiple sclerosis (MS), |
| | paclitaxel versus saline | viruses (e.g., West Nile, |
| | treated subjects | herpes, HIV, and influenza), |
| | | traumatic brain injury |
| | | (TBI), chronic traumatic |
| | | encephalopathy (CTE) |
| Edema (acute) | T2-mapping on MRI | TBI |
| | showing a significantly | |
| | reduced injury-associated | |
| | edema in a mouse model of | |
| | TBI as compared to saline | |
| | treated subjects | |
| Structural | T1-weighted MRI | TBI |
| repair/preservation | indicating reduced injury | |
| | volume in paclitaxel versus | |
| | saline treatment | |
| Myelin preservation | Macromolecular proton | TBI, MS |
| | fraction MRI (MPF | |
| | imaging) indicating | |
| | preservation of myelin on | |
| | injury border | |
| Tau-hyperphosphorylation | Immunostain in the CA1 | TBI, AD, CTE, |
| | region of the hippocampus | frontotemporal lobe |
| | of 3xTg-AD mice (7.5 mos.) | dementia (FTD) |
| | showing decreased | |
| | evidence of phosphorylated | |
| | neuronal cell bodies using | |
| | antibody recognizing | |
| | phosphor tau at threonine | |
| | 181. Number of phosphor- | |
| | neurons can be evaluated | |
| | by blinded rater | |
| Neurological function - gait | Improved gait parameters | TBI |
| (short term, 8 days) | can be evaluated by | |
| | Catwalk automated gait | |
| | analysis (Noldus) by | |
| | paclitaxel over saline | |
| | treated subjects | |
| Neurological function - foot | Grid test showing | TBI |
| faults (long term, 32 days) | improvement in number of | |
| | contralateral foot faults at | |
| | 32 days post injury with | |
| | paclitaxel | |
| Cognitive function/memory | Paclitaxel treated subjects | TBI, AD, CTE |
| short and long term | showing the same degree of | |
| | learning and memory as | |
| | sham (no TBI) subjects in | |
| | the radial water tread maze | |

### Formulations

The taxanes can be administered in the form of pharmaceutical compositions which is a combination of a taxane and a pharmaceutically acceptable carrier. These compositions can be prepared in a manner well known in the pharmaceutical art. Pharmaceutical compositions and formulations for intranasal administration may include drops, sprays, liquids, and powders. Conventional pharmaceutical carriers, aqueous, powder or oily bases, thickeners and the like may be necessary or desirable.

In making the compositions of the invention, the taxane is typically mixed with an excipient. When the excipient serves as a diluent, it can be a solid, a semisolid, or liquid material, which acts as a vehicle, carrier or medium for the taxane. Thus, the compositions can be in the form of suspensions, emulsions, solutions, aerosols, ointments, or powders, containing, for example, up to 10 percent by weight of the taxane in a sterile solution.

The compositions can be formulated in a unit dosage form, each dosage containing from about 5 to about 100 mg, more usually about 10 to about 30 mg, of the taxane. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, compositions in can be nebulized by use of inert gases. Nebulized solutions may be breathed directly from the nebulizing device or the nebulizing device can be attached to a face mask, face tent, or intermittent positive pressure breathing machine.

The following Examples are included for the purpose of illustrating, not limiting, the described embodiments. Example 1 describes a general protocol for assessing the effectiveness of microtubule-stabilizing drugs in a rodent model. Example 2 describes an evaluation of intranasal delivery of paclitaxel in a rodent model of traumatic brain injury. Example 3 describes the intranasal administration of taxane for Alzheimer's disease treatment. Example 4 investigates the effect of taxol on axonal transport rates and on astrocyte activation.

### EXAMPLES

### Example 1. General Protocol for Evaluating Taxane Effectiveness in Treatment of Alzheimer's Disease or Alzheimer-Related Tauopathies

This Example provides a general protocol for evaluating the efficacy of intranasal delivery of microtubule-stabilizing drugs to improve outcome following AD and Alzheimer-related tauopathies. The protocol can be easily translated to human patient evaluations.

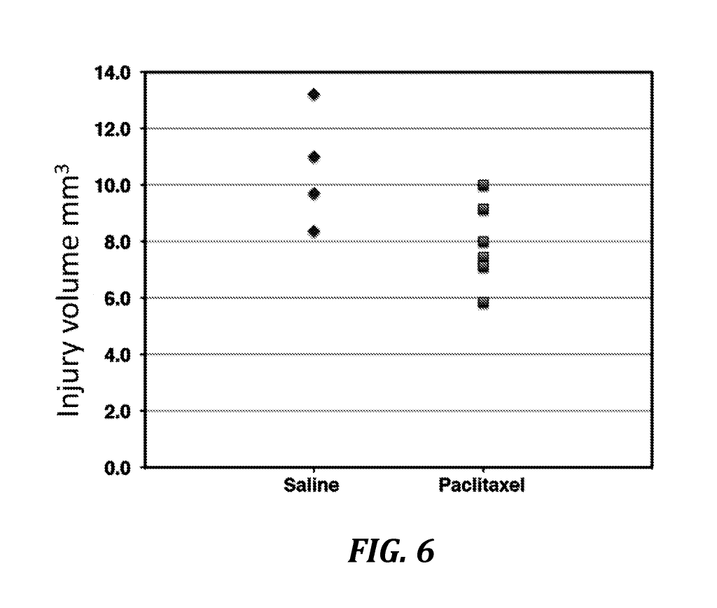

A flow chart of the general protocol for evaluating microtubule-stabilizing drugs in the treatment of AD and Alzheimer-related tauopathies is shown in FIG. 5 and is described below.

Prior to the concussive injury, rodents receive baseline assessment of cortical axonal transport rates and axonal integrity using manganese-enhanced magnetic resonance imaging of axonal transport (AT-MEMRI) and diffusion tensor imaging (DTI) of white matter integrity. The use of in vivo imaging permits an objective, quantifiable and longitudinal measure of drug treatment effectiveness that is directly translatable to a human clinical study. Baseline behavioral measures of memory and motor function are also assessed.

After concussive injury, rats are treated with a commercially available taxol (paclitaxel) using intranasal lavage—dosages and concentrations can vary based upon effectiveness. Control groups receive saline lavage. The effectiveness of the taxane to treat concussive type injuries is assessed in vivo using AT-MEMRI, flurorodeoxyglucose-positron emission tomography (FDG-PET), and DTI as behavior indicators. The effectiveness is also assessed using ex vivo methods such as histological examination for damage to white matter tracks.

Positive indicators of effectiveness in response to the intranasal administration of taxanes can include any or all of the following: 1) decreased recovery time to baseline of in vivo measures of axonal transport, structure and behavior, 2) improved total recovery should return to baseline not occur, 3) reduction in potential long term effects of repeat concussive injuries, and/or 4) reduction in histological evidence of axonal injury.

Rodent models are also pretreated with intranasal taxanes prior to concussive injury to assess if brains are more “injury-resistant”.

A variety of tests can be carried out on the rodent models and are discussed below.

### Radial Water Tread Maze

The radial water tread maze tests spatial (hippocampal) learning and memory. The apparatus consists of a 32″ steel circular enclosure with 9 holes (8 decoy and 1 exit) positioned 1½″ above the apparatus floor. The exit hole leads to a ‘safety box’ (a small, heated, dark box). Five large visual cues line the sides of the apparatus. Before each mouse, the maze is sanitized with 70 percent ethanol and filled with 1″ of cold water (12-14 C). Water is changed between mice and temperature monitored to ensure the desired range. The mouse is placed in the center of the maze and has 180 s to find the ‘safety box’. If the mouse fails to find the exit the trial is recorded as 180 s and the mouse is led to the exit by hand. Once inside the ‘safety box’, the mouse was given an edible reward and allowed to remain in the box for one minute. Then mouse is removed and box and maze resanitized with 70 percent ethanol for the second trial. Each mouse receives 3 trials/day, with a 1 min rest interval. Mice are given 3 trials/day during a 4-day acquisition period. On the 5th day, mice are given a short-term memory test consisting of one series of 3 trials. Mice are tested again one week after their short-term memory test (day 12) as a long-term memory test. All trials are averaged by day, and a lower value (in seconds) represents a greater ability to form and store spatial memories.

### Novel Object Recognition

Mice are habituated for 50 minutes in an open field. The next day, mice are presented with 2 novel objects for 6 mins. Retention is tested at 1, 3, 6 and 24 hrs after the initial exposure, by placing one familiar and one novel object in the open field and measuring percentage of time spent in proximity to each. A video tracking system is used.

### Elevated-Plus Maze

The elevated-plus maze measures anxiety, exploration and activity in mice by taking advantage of their tendency to avoid open and elevated areas. The maze consists of a central square (5×5 cm), with 4 radiating arms. Two of arms (closed) have plexiglass walls (15×m high), and the other 2 do not have walls (open arms), but have a 0.25 cm edge to prevent the mice from leaving. The maze is elevated 45 cm above the floor. A video tracking system is used to measure entries and duration in the center, open and closed arms. Mice are placed in the central square of the apparatus, facing an open arm. Mice are allowed to explore the apparatus for 5 mins while data are collected, including line crosses, rears, head dips, grooming, stretch attend postures, urination puddles, fecal boli, closed-arm entries and duration, open-arm entries and duration, center entries and duration.

### Imaging

MRI: Mice are anesthetized with isoflurane and scanned on an ultra-high resolution 14T MRI (Avance III, Bruker BioSpin Corp, Billerica, Mass.). Dynamic manganese enhanced magnetic resonance imaging (dyMEMRI): Mice are anesthetized with isoflurane and administered a unilateral injection of Sul of 1 M MnCl₂ intranasally (with occlusion to block septal window). Parameters for magnetization prepared rapid gradient echo (MPRAGE) (TR/TE=11/5.3 ms; Ti=1000 ms; FA=9 deg acquired matrix 108×108 mm over 55 slices, voxel 0.2×0.2×0.4 mm3 interpolated to 0.1×0.1×0.2 mm3). Mice are scanned dynamically for 45 min (early uptake) at 1 hour post administration, allowed to recover and scanned again at 4-6 hours post (late transport) for 60 min. DTI acquired as 4 shot echo planar image (EPI); TR/TE=4000/18 ms, 30 diffusion directions, multislice 2D, b=1000 s/mm2, FOV 19×19 mm, matrix size=128×128 with slice thickness=0.5 mm over 9 min. MicroPET: Metabolic brain activity is assessed using FDG-PET performed under isoflurane anesthesia with 30 min uptake after 250 μCi intraperitoneal injection of FDG. High-resolution images are acquired over the whole brain for 30 min with 3D ordered subsest expectation maximization-maximum aposteriori (OSEM/MAP) reconstruction. Spatial resolution using 3D OSEM/MAP is approximately 1 mm. For PET imaging of tau protein accumulation, ['⁸F]-THK523 is produced and radiolabeled using known methods. PET imaging follows the same protocol as outlined for FDG. Image analysis: Automated programs for image analysis (NEUROSTAT, U of Wash) in which image sets are co-registered and stereotactically aligned to the mouse atlas are used. Using Neurostat/3D-SSP, global-normalized cerebral metabolic rate of glucose (CMRg1u) values are analyzed via two complementary methods: (1) whole brain (WB) voxelwise analyses to evaluate: (a) Between group differences at each voxel (using one-tail t-statistics transformed to z-scores via a probability integral transformation and a significance threshold based on a random Gaussian field and Euler characteristic to control the Type I error rate at p=0.05 (Z=4.0)); and (b) Within group voxel-by-voxel correlations (Pearson's r) between CMRglu and other outcome measures, following transformation of r values to Z-scores; and (2) VOI-based analyses to evaluate AD effects on specific brain regions, based on mean CMRg1u values within predefined anatomical

VOIs, which can then be used as dependent variables in a variety of statistical analyses.

Histology and Westerns. All confocal microscopy and immunohistochemistry are performed on perfusion fixed tissue prepared from animals that, immediately after euthanasia with pentobaritol (100 mg/kg), are perfused with saline followed by 4 percent paraformaldehyde/saline. For biochemical experiments (Western blots, etc.) other animals are perfused after death with saline and then the brains are rapidly dissected and flash frozen in liquid N2. Brain sections corresponding to the olfactory bulb are stained for Fluoro-Jade (1:1000, Histo-Chem Inc., AR, USA) as a marker of degenerating neurons and can be used to assess if 9 months of paclitaxel intranasal treatment may be neurotoxic. Glial fibrillary acidic protein (GFAP, 1:1500, Dako, Carpinteria, Calif.), an astroglial marker, and ionized calcium-binding adapter molecule 1 (Iba-1) (1:1000 Dako, Carpinteria, Calif.), a marker of microglial activation are processed using Avidin-Biotin procedure, which uses biotinylated secondary antibodies, avidin coupled to horse radish peroxidase (HRP) and reacted with 3,3′diaminobenzidine.

### Tau Phosphorylation

Sections are incubated with the AT8 antibody (Ser202; Pierce, Rockford, Ill.), followed by FITC-conjugated anti-rat IgG (Vector Laboratories). AB: immunostain with 6e10 human anti-AB monoclonal antibody.

### Stereology

Sections are analyzed with the optical dissector, using an Olympus BH2 microscope with a digital color camera attached to a DataCell computer assisted image analysis system.

### Aβ Levels

Brains are homogenized with buffer (5 M guanidine-HCl and PBS, pH 8.0) with 1× protease inhibitor (Calbiochem, San Diego, Calif.), mixed for 3 hrs at room temperature, centrifuged at 16,000×g for 20 min at 4° C. and resulting supernatants are diluted 10× in Dulbecco's PBS, (pH 7.4, 5 percent bovine serum albumin and 0.03 Tween 20). Aβ 1-42 uses commercially available sandwich-type ELISA (Biosource International, Camarillo, Calif.).

### Western Blot

Cytosolic and particulate fractions are assayed by the Lowry method, loaded into 10 percent SDS-PAGE gels, blotted onto nitrocellulose paper and incubated with antibodies against; 1) phosphorylated amyloid precursor protein (APP) (APP-p) (Thr668, 1:1200; Cell Signaling Technology, Beverly, Mass.) 2) full-length (FL) APP (mouse monoclonal, clone 22C11, 1:20,000; Chemicon, Temecula, Calif.), AB (mouse monoclonal, clone 6E10, 1:1000; Signet Laboratories, Dedham, Mass.), APP C-terminal fragments, neprilysin (mouse monoclonal, clone CD10, 1:1000; Abcam, Cambridge, Mass.), and beta-secretase 1 (BACE1) (1:1000; ProSci, Poway, Calif.), followed by secondary antibodies tagged with HRP. Samples are visualized by enhanced chemiluminescence and analyzed by a Versadoc XL apparatus.

### Example 2. Evaluation of Cytoskeletal Stabilization Therapy for Traumatic Brain Injury

Rodent subjects (C57BL6 mice, 10 wks, male, n=12) had craniotomy over the right frontoparietal cortex of 5 mm, plus mild controlled cortical impact (CCI) surgery using a pneumatic impactor (AmScien Instruments, Richmond, Va.) at 6 m/s strike velocity, 1 mm depth of penetration, and 150 ms contact time, under isoflurane anesthesia. Immediately following CCI, 200 ug/kg paclitaxel (n=6) or vehicle (n=6) was applied to the brain injury site. Sham surgery (craniotomy, but no CCI) was performed on controls (n=3).

At 2 days post-surgery, gait assessment of the subjects was conducted using CatWalk automated gait analysis (Noldus Information Tech, The Netherlands) followed by high-tesla magnetic resonance imaging (14T MR Avance III Ultrashield, Bruker BioSpin, Billerica, Mass.). T1-weighted and quantitative T2 maps were obtained: MDEFT (3D modified driven equilibrium Fourier transform), Fractional anisotropy: 12°, TR (repetition time): 5000 ms, TE (echo time): 1.9ms, resolution 0.140×0.140×0.25 mm³, 64 slices; T2 map: TR=2000 ms, 16 echoes, spacing:6.7 ms, TE1: 6.7 ms, TE 2:13.4 ms, resolution 0.12×0.12×1.0 mm³, 15 slices. Manual volume of interest (VOI) analysis of injury volume and volume of edema related to injury was performed.

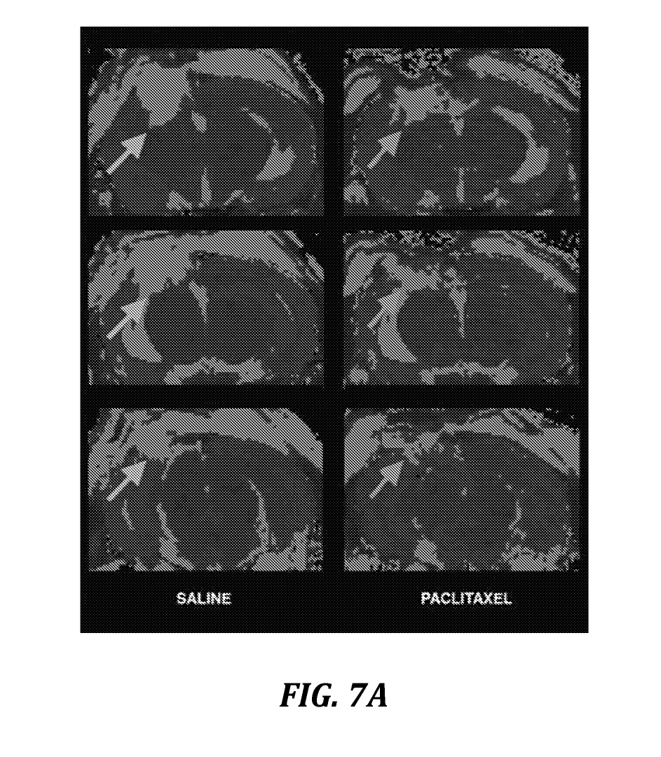

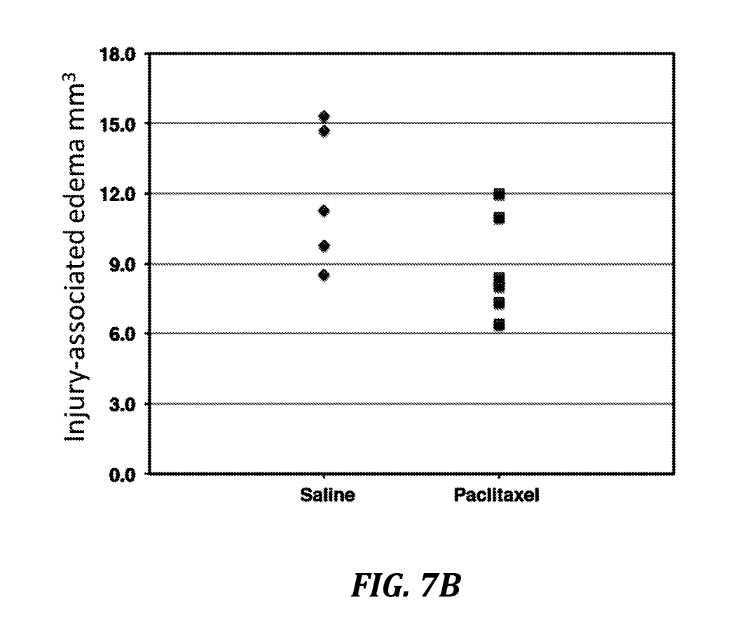

Referring to FIG. 6, injury analysis on T2 and T1 images, blinded to therapeutic regimen, indicated a 20 percent reduction in injury volume with paclitaxel treatment (9.96±2.3 versus 7.94±1.5mm³, p≦0.05). Referring to FIGS. 7A and 7B, hyperintense voxels (edema) on quantitative T2 maps were reduced by 26 percent (11.92±3.0 versus 8.86±2.2mm³, p≦0.05).

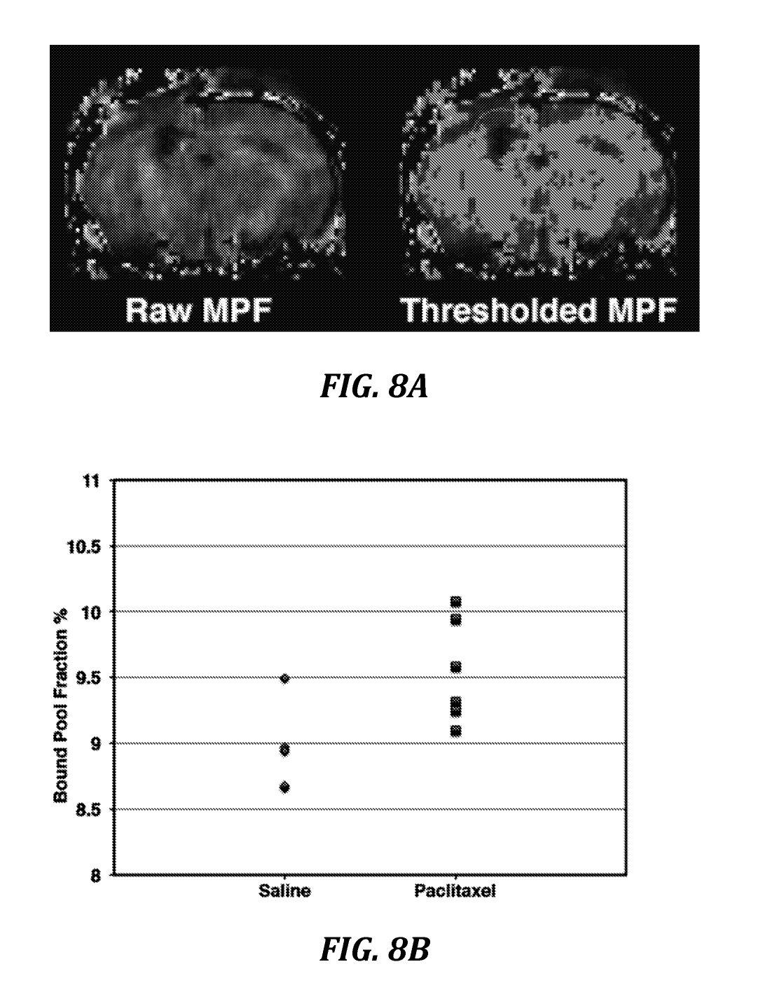

Macromolecular proton fraction (MPF) imaging is a quantitative magnetic resonance technique that measures the magnetization transfer between protons bound to water and protons bound to macromolecules. MPF imaging was performed to evaluate myelin degradation adjacent to injury. FIG. 8A shows a raw MPF image, thresholded to mask out injury pixels. Mean myelin density was calculated for a region of interest (circular region having a diameter of 0.5 mm) that was larger than the actual injury (1.5 mm). As shown in FIG. 8B, paclitaxel preserves myelin density around injury after CCI as MPF bound percent was significantly increased on the injury side by 6.6 percent in paclitaxel treated subjects (9.45±0.4 vs. 8.95±0.3, mn±sd, p≦0.05). No difference was seen in the contralateral cortical area.

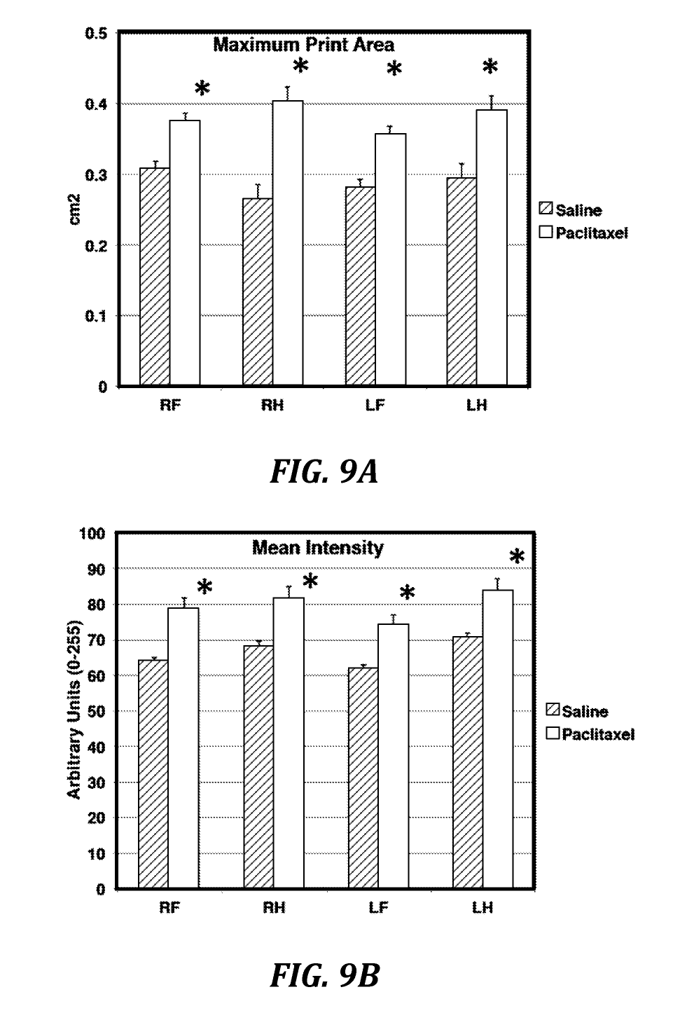

Referring to FIGS. 9A and 9B, paclitaxel resulted in improved gait (computer-recorded objective analysis) for maximum print area (0.38±0.09 versus 0.29±0.08 cm², p≦0.05) and mean intensity (79.45±14.26 versus 66.38±5.52, p≦0.05) over vehicle group. Improvements in several indices, including maximum print area (22-52 percent increased), mean intensity (19-22 percent increased), print area (20-45 percent increased), print width (5-17 percent increased), print length (12 percent increase, right hind (RH) only), and swing (12 percent decreased, right front (RF) and left hind (LF) only).

The MPF results were corroborated using cross-relaxation imaging. Cross-relaxation imaging (CRI) is a quantitative magnetic resonance technique that measures the kinetic parameters of magnetization transfer between protons bound to water and protons bound to macromolecules. Here, in vivo, four-parameter CRI of normal rat brains (n=5) at 3.0 T was first directly compared to histology. The bound pool fraction, f, was strongly associated with myelin density (Pearson's r=0.99, p<0.001). The correlation persisted in separate analyses of gray matter (GM; r=0.89, p=0.046) and white matter (WM; r=0.97, p=0.029). The CRI results validated the MPF results, in that the taxane helped to preserve myelin density around the injury. Fractional anisotropy imaging was carried out to evaluate the integrity of underlying external capsule. No significant improvement was found in the underlying white matter integrity following CCI with administration of paclitaxel. CCI surgery for both treatment groups caused decreased integrity in the external capsule. Although CCI surgery resulted in significant (and nearly significant) decreased FA values in the external capsule compared to shams, paclitaxel did not result in improvement. There may be several reasons: 1) DTI imaging was sufficiently sensitive and the SNR was too high to detect subtle improvements, 2) drug did not penetrate that deep into the tissue, 3) possibly a different parameter such as radial diffusivity may be more sensitive, 4) timing of imaging compared to hypothesized therapeutic effect may be not optimized for this outcome, or 5) paclitaxel neuroprotective/neurotherapeutic effects act in ways other than maintaining cytoskeletal integrity. While the paclitaxel was not intranasally administered for fractional anisotropy imaging, this result shows a therapeutic effect of the taxane after TBI.

The results indicate that intranasally administering taxanes to stabilize axonal cytoskeleton following TBI improved outcome in neurological/gait assessment and demonstrated improvement on MR imaging biomarkers. This improvement appears to be mediated by reductions in size of injury and corresponding post-injury edema.

### Example 3. Intranasal Administration of Taxane for Alzheimer's Disease Treatment

The effect of intra-nasal treatment of taxanes on the development of neurodegenerative pathology in a model of Alzheimer's disease was also studied. Using a similar study design as above (without the concussive injury component), transgenic 3×Tg-AD mice were treated with intranasal taxanes (paclitaxel, 0.6 mg/kg) at two week intervals for 3 months during the time when pathology is developing. Treatment effectiveness was assessed with the same outcome measures as described in Example 1.

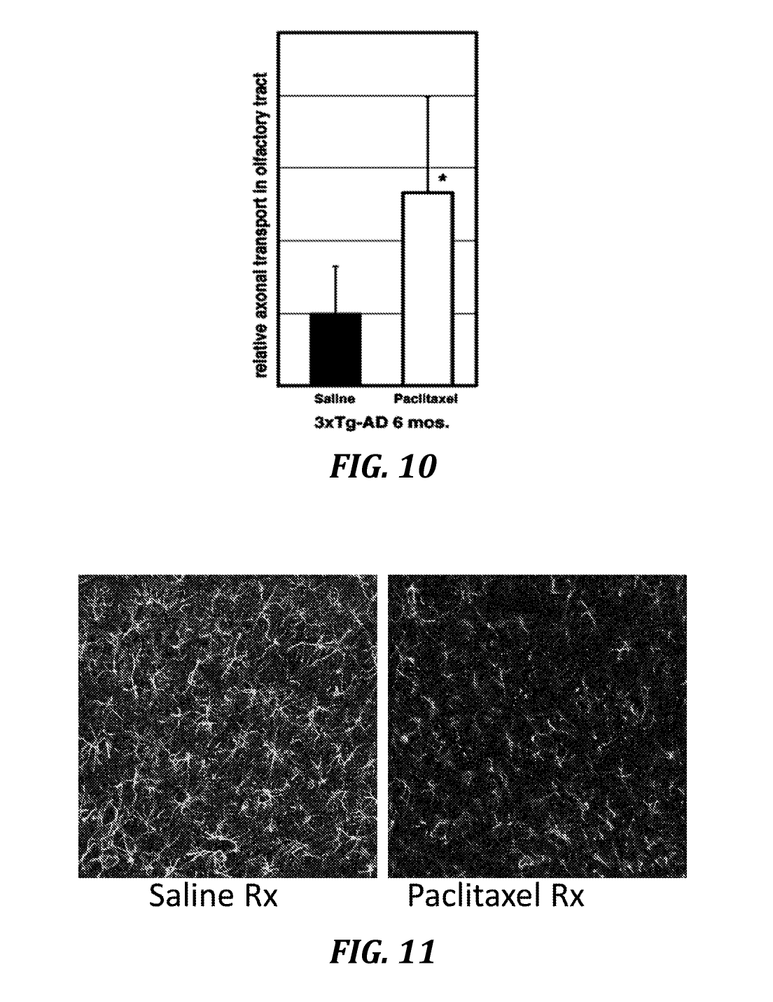

Intranasal paclitaxel (Hospira, Inc., Lake Forest, Ill.) was administered to 3×Tg-AD mice (0.6 mg/kg in 0.9 percent saline, 5 μA per nostril). Referring to FIG. 10, imaging using manganese-enhanced MRI (MEMRI) to assess axonal transport rates in the olfactory tract in vivo indicated that paclitaxel intranasal administration increased transport significantly in 3×Tg-AD mice. Paramagnetic, MnCl₂ 1M solution was administered intranasally. Mn²⁺, a calcium analog was taken up by neurons and transported via axonal transport processes along tracts. Dynamic T1-weighted MR imaging detected signal enhancement and post-processing algorithms calculate a relative rate of transport. Details of the imaging technology were described in Cross et al., Neuroimage. 39, 915-26.

Preliminary data indicated that 3×Tg-AD mice have significantly reduced fractional anisotropy (FA), a marker of white matter integrity as early as 3 months of age (3 month Wild-type: 0.38±0.02 vs. 3 month 3×Tg-AD: 0.32±0.03, 16 percent decreased, p≦0.01). FDG-PET and automated voxelwise image analysis can also be used to assess similar changes and response to paclitaxel treatment. Previous research indicated that white matter structural integrity (assessed by DTI) was strongly associated with hypometabolic regions (assessed by FDG-PET) in normal aging and MCI patients, as described in Cross et al., J. Nucl. Med. 54, 1278-84.

### Example 4. Investigation of Effect of Taxol on Axonal Transport Rates and on Astrocyte Activation

Intranasal administration of paclitaxel to 3×Tg-AD mice was performed according to Example 2. Improved axonal transport rates and decreased evidence of activated astrocytes was observed. These findings suggest that paclitaxel asserts a positive effect on neuronal function and reduces overall injury that may be related to effects beyond MT stabilization.

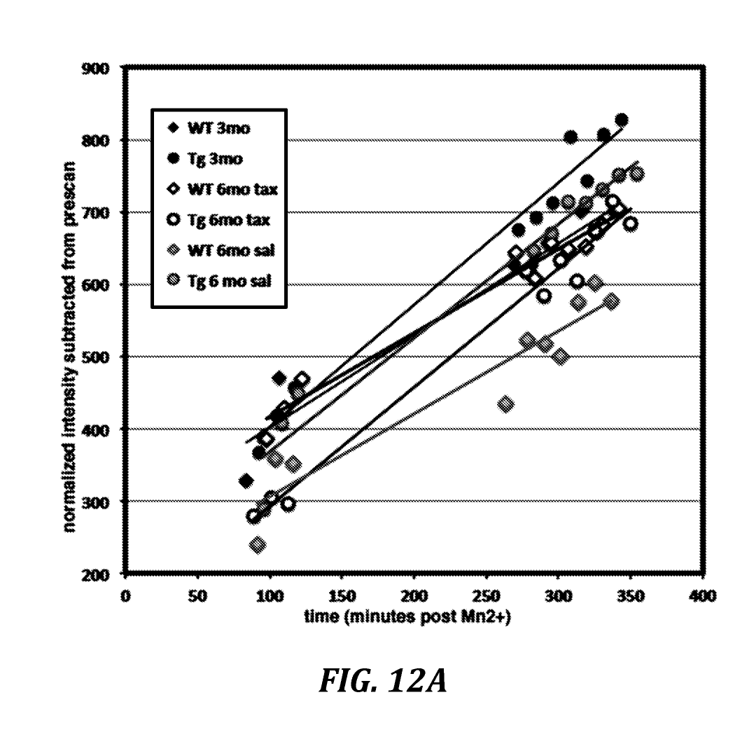

Preliminary data suggest that paclitaxel may reduce neuroinflammatory response from TBI (T2-maps indicated reduced edema, FIGS. 7A and 7B). The application of paclitaxel to 3×Tg-AD mice (0.6 mg/kg at 2 week intervals for 3 months, intranasal administration) was investigated. It has been shown that microglia and astrocytes respond acutely to brain injury. Here, histology of 3×Tg-AD brains indicated markedly reduced reactive astrocytes as evidenced by decreased glia fibrillary acidic protein (GFAP) expression, which is indicative of the inflammatory state in the CAl region of the hippocampus. FIG. 11 shows a representative example from confocal microscopic examination of the CAl region of the hippocampus from 4 saline and 5 paclitaxel-treated 3×-Tg-AD mice. The images of FIG. 11 showed hippocampus samples immunostained with GFAP (Millipore) for astrocytes (grey) that were maximum-field projections compiled from 20 z-plane images acquired at 2 μm intervals on a confocal microscope (Leica). The paclitaxel-treated and saline-treated samples: (i) were imaged together in one session; (ii) using identical acquisition parameters; and (iii) post-processed using only linear brightness and contrast adjustments applied identically.

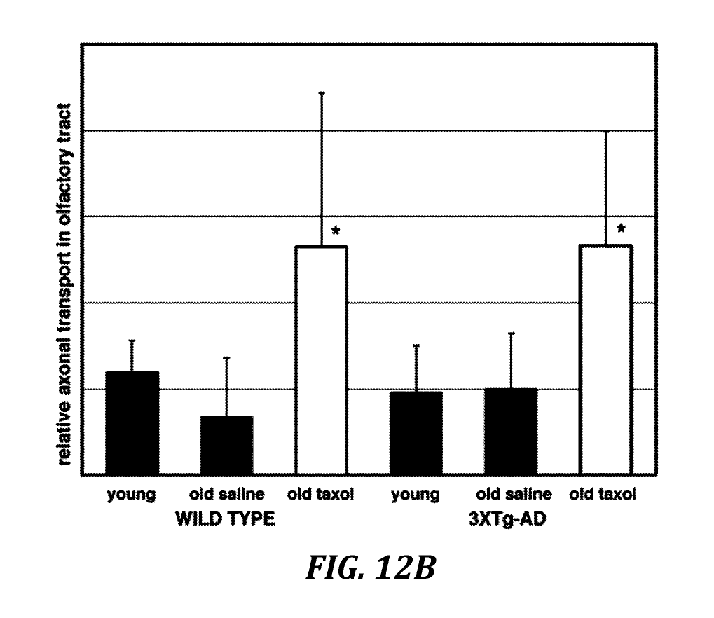

FIGS. 12A and 12B show results from preliminary analysis of axonal transport in paclitaxel-treated triple transgenic mice compared to wild type controls. Referring to FIG. 12A, in the olfactory nucleus, young 3×Tg-AD had an increased slope for time/intensity compared to wild-type (1.69±0.47 versus 1.26±0.46 for 3×Tg-AD versus wild-type, respectively, p=0.03), possibly due to Ca²⁺ dysregulation). Referring to FIG. 12B, transport rates were relatively increased in both 3×Tg-AD and wild-type mice treated with paclitaxel compared to young wild-type controls (*p≦0.01).

The results above indicate that paclitaxel reduced GFAP expression levels, which is consistent with the idea that paclitaxel may influence the basal inflammatory state of the CNS in these mice.

While illustrative embodiments have been illustrated and described, it will be appreciated that various changes can be made therein without departing from the spirit and scope of the invention.

1-27. (canceled)

28. A method of increasing microtubule stabilization in a brain tissue of a subject, of decreasing tau protein oligomerization in a brain tissue of a subject, of decreasing aggregation of a hyperphosphorylated tau protein in a brain tissue of a subject, of ameliorating a condition having decreased microtubule stabilization in a brain tissue of a subject, of ameliorating a condition having tau protein oligomerization in a brain tissue of a subject, of ameliorating a condition having a hyperphosphorylated tau protein in a brain tissue of a subject, or of ameliorating a condition having neuroinflammation in a brain tissue of a subject, comprising intranasally administering to the subject a therapeutically effective amount of a taxane.

29. The method of claim 28, wherein intranasally administering comprises an intranasal lavage or administering an aerosol to a nasal passage.

30. The method of claim 28, wherein the taxane is selected from the group consisting of paclitaxel and docetaxel.

31. The method of claim 28, wherein the taxane is a crystalline paclitaxel.

32. A method of treating Alzheimer's disease in a subject or of treating an Alzheimer-related tauopathy in a subject, comprising intranasally administering to the subject a therapeutically effective amount of a taxane.

33. The method of claim 32, wherein the Alzheimer's disease or Alzheimer-related tauopathy is each characterized by aggregation of a hyperphosphorylated tau protein in brain tissue into bundles of filaments.

34. The method of claim 32, wherein the Alzheimer-related tauopathy is selected from the group consisting of Lytico-Bodig disease, tangle-predominant dementia, ganglioglioma, fronto-temporal dementia and Parkinsonism linked to chromosome 17 (FTDP-17) caused by tau mutations, Pick disease, corticobasal degeneration, and progressive supranuclear palsy.

35. The method of claim 32, wherein intranasally administering comprises an intranasal lavage or administering an aerosol to a nasal passage.

36. The method of claim 32, wherein the taxane is selected from the group consisting of paclitaxel and docetaxel.

37. The method of claim 32, wherein the taxane is a crystalline paclitaxel.

38. A method of treating a traumatic brain injury in a subject, treating a chronic traumatic encephalopathy in a subject, or decreasing risk for an onset of Alzheimer's disease or chronic traumatic encephalopathy in a subject, comprising intranasally administering to the subject a therapeutically effective amount of a taxane.

39. The method of claim 38, wherein the traumatic brain injury comprises skull fracture, brain swelling, penetrating skull injury, concussion resulting in loss of consciousness, post-concussive symptoms, or any combination thereof

40. The method of claim 39, wherein the post-concussive symptoms comprise a headache, mental fog, decreased attention, decreased reaction time, concentration, sleep disturbance, mild motor disturbance, or any combination thereof.

41. The method of claim 38, wherein the traumatic brain injury comprises an acute traumatic brain injury.

42. The method of claim 38, wherein the traumatic brain injury is a concussion.

43. The method of claim 38, wherein intranasally administering comprises an intranasal lavage or administering an aerosol to a nasal passage.

44. The method of claim 38, wherein the taxane is selected from the group consisting of paclitaxel and docetaxel.

45. The method of claim 38, wherein the taxane is crystalline paclitaxel.

46. The method of claim 38, wherein the taxane is administered in an amount of 0.3 mg/kg to about 2 mg/kg per dose.

47. The method of claim 46, wherein the dose is repeated at every two weeks.
